(19) United States
(12) Patent Application Publication
(10) Pub. No.: US 2017/0312414 A1
(43) Pub. Date: Nov. 2, 2017

(54) DIALYSIS-FLUID SUPPLY SYSTEM

(71) Applicant: NIKKISO CO., LTD., Tokyo (JP)

(72) Inventors: Masato FUJIWARA, Higashimurayama-shi (JP); Yoshimichi MASUDA, Higashimurayama-shi (JP); Hiroshi NIMURA, Higashimurayama-shi (JP); Manabu YOKOMICHI, Higashimurayama-shi (JP)

(73) Assignee: Nikkiso Co., Ltd., Tokyo (JP)

(21) Appl. No.: 15/524,538

(22) PCT Filed: Nov. 13, 2015
(86) PCT No.: PCT/JP2015/082024

§ 371 Date: May 4, 2017

(30) Foreign Application Priority Data
Nov. 20, 2014 (JP) 2014-235458

(51) Int. Cl. A61M 1/16; A61M 1/36; A61K 33/00; A61K 47/12; A61K 33/14; A61K 31/19

(52) U.S. Cl. CPC A61M 1/1666; A61K 33/14; A61M 1/3692; A61K 31/19; A61K 47/12; A61M 1/1668; A61K 33/00; A61M 2205/3379; A61M 2205/3393; A61M 2209/10

(57) ABSTRACT

This dialysis-fluid supply system, which mixes a diluent and at least two drugs to generate a dialysis fluid, and outputs said dialysis fluid, is provided with: a mixing tank which mixes the drugs and the diluent to generate the dialysis fluid; a storage tank which stores and outputs the dialysis fluid generated by the mixing tank; a transport mechanism for transporting, to the storage tank, the dialysis fluid generated by the mixing tank; an output mechanism for outputting, to a dialysis device, the dialysis fluid stored in the storage tank; and a control unit for controlling the driving of the transport mechanism and the output mechanism.

### TECHNICAL FIELD

The present invention relates to a dialysis-fluid supply system that generates dialysis fluid by mixing a diluent and at least two drugs and outputs the dialysis fluid.

### BACKGROUND ART

In a conventionally known dialysis-fluid supply system, a plurality of drugs and a diluent (for example, water) are mixed together to generate and output dialysis fluid. Widely known examples of the dialysis-fluid supply system include a dialysis-fluid continuous supply system that generates dialysis fluid by continuously mixing a plurality of drugs and a diluent. The dialysis-fluid continuous supply system requires a measurement pump capable of accurately measuring the amounts of a diluent and a drug concentrated solution transferred for mixing. However, such a measurement pump capable of performing accurate measurement is typically expensive and requires frequent maintenance.

To avoid this problem, in a batch system, the drugs and the diluent in amounts necessary for achieving a desired concentration are collectively supplied to a tank to generate dialysis fluid, instead of continuously generating dialysis fluid. Patent Literatures 1 and 2 disclose such dialysis-fluid batch supply systems. In the batch system, drugs and the like are measured in advance, or a measurement means is provided in the tank, which eliminates the need to provide an accurate measurement pump. This leads to further reduction in the price of the dialysis-fluid supply system and also in the amount of maintenance work.

### CITATION LIST

### Patent Literature

Patent Literature 1: National Publication of International Patent Application No. 2008-526375

Patent Literature 2: Japanese Patent Laid-Open Publication No. Hei 9-618

Patent Literature 3: U.S. Pat. No. 4,134,834

### SUMMARY OF INVENTION

### Technical Problem

However, the dialysis-fluid supply system disclosed in Patent Literature 1 is intended to employ lactic acid dialysis fluid used in peritoneal dialysis, but not bicarbonate dialysis fluid widely used in blood dialysis treatment nowadays. The dialysis-fluid supply system disclosed in Patent Literature 2 is intended to employ bicarbonate dialysis fluid, but can be used for dialysis treatment in a relatively short time (for example, two hours). Specifically, bicarbonate dialysis fluid is generated by mixing a diluent and two kinds of drugs called drugs A and B. However, when a certain time (for example, two hours) has elapsed after the mixing of the drugs A and B, a deposit is generated in this bicarbonate dialysis fluid, and the bicarbonate dialysis fluid can no longer be used in blood dialysis treatment. Thus, in the technology of Patent Literature 2, in which dialysis fluid is generated in one tank, the dialysis fluid can be output only in an amount that can be used up within this certain time. However, a continuous dialysis treatment in a longer time (for example, six hours) required depending on the condition and lifestyle of a patient, and such a requirement cannot be sufficiently met by the technology of Patent Literature 2.

Patent Literature 3 discloses a system including two tanks. In this system, while one of the tanks outputs dialysis fluid, the other tank generates dialysis fluid, and when the one tank runs out of the dialysis fluid, the other tank outputs the dialysis fluid while the one tank generates dialysis fluid. However, the dialysis-fluid supply system of Patent Literature 3 is not intended to employ bicarbonate dialysis fluid. Moreover, the dialysis-fluid supply system of Patent Literature 3 includes a measurement pump to measure a diluent, resulting in an expensive system that requires frequent maintenance work. In addition, in the technology of Patent Literature 3, switching is performed between a tank that generates dialysis fluid and a tank that outputs the dialysis fluid. Thus, in the technology of Patent Literature 3, the two tanks each need to function as the dialysis-fluid generation tank and the dialysis-fluid storage tank. With this configuration, the technology of Patent Literature 3 requires, for the two tanks, two sets of valves, measurement pumps, drug supply mechanisms, and the like, which is wasteful.

It is an advantage of the present invention to provide a dialysis-fluid supply system that generates and outputs dialysis fluid obtained by mixing a plurality of drugs, and is inexpensive and capable of outputting a large amount of the dialysis fluid.

### Solution to Problem

A dialysis-fluid supply system according to the present invention is a dialysis-fluid supply system that generates dialysis fluid by mixing a diluent and at least two drugs, and outputs the dialysis fluid. The dialysis-fluid supply system includes: a mixing tank that generates dialysis fluid by mixing the drugs and the diluent; a storage tank that stores therein the dialysis fluid generated by the mixing tank and outputs the dialysis fluid; a transfer mechanism that transfers the dialysis fluid generated by the mixing tank to the storage tank; an output mechanism that outputs the dialysis fluid stored in the storage tank to a dialysis device; and a control unit that controls driving of the transfer mechanism and the output mechanism.

In a preferable aspect, the control unit may generate dialysis fluid in the mixing tank while outputting dialysis fluid froth the storage tank to a dialysis device. In another preferable aspect, the mixing tank may include a weight sensor that measures the weight of supplied fluid or a level sensor that measures the level of supplied fluid, and the control unit may monitor the supply amount of each drug or the diluent to the mixing tank based on a result of detection by the weight sensor or the level sensor.

In another preferable aspect, the dialysis-fluid supply system may further include a circulation mechanism that internally and externally circulates fluid inside the mixing tank to perform agitation. In another preferable aspect, the capacity of the storage tank may be larger than the capacity of the mixing tank. In another preferable aspect, the control unit may execute a cleaning process of cleaning the mixing tank and the storage tank by causing cleaning fluid to flow through the mixing tank and the storage tank in this order.

### Advantageous Effects of Invention

In a dialysis-fluid supply system according to the present invention, dialysis fluid is generated by a mixing tank and stored in a storage tank for outputting, which allows continuous generation and outputting of the dialysis fluid for a long time without a measurement pump. Accordingly, the dialysis-fluid supply system is inexpensive and is capable of outputting a large amount of dialysis fluid compared to a conventional dialysis-fluid supply system.

### BRIEF DESCRIPTION OF DRAWINGS

FIG. 1 is a diagram illustrating the configuration of a dialysis system according to an embodiment of the present invention.

FIG. 2 is a timing chart illustrating timing of opening and closing of valves in a dialysis-fluid supply system.

### DESCRIPTION OF EMBODIMENT

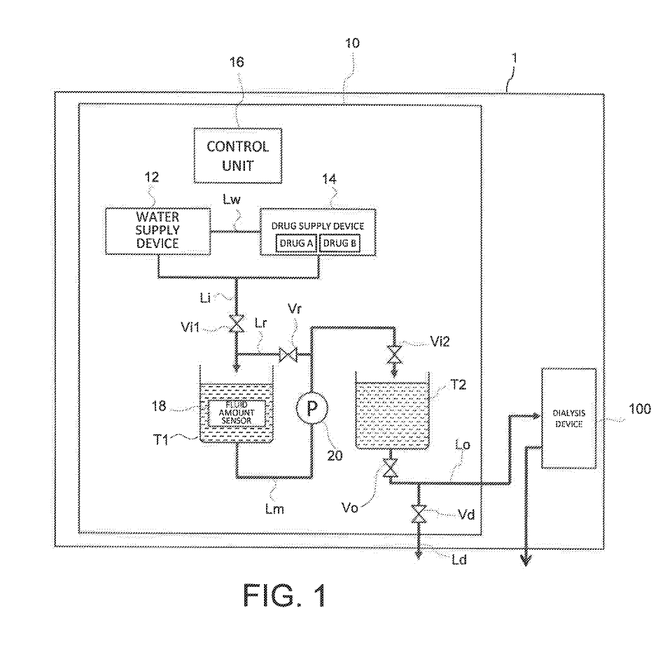

The following describes a dialysis system 1 according to an embodiment of the present invention with reference to the accompanying drawings. FIG. 1 illustrates the configuration of a dialysis-fluid supply system 10 according to the embodiment of the present invention. The dialysis system 1 illustrated in FIG. 1 includes the dialysis-fluid supply system 10 and a dialysis device 100 connected with the dialysis-fluid supply system 10. The dialysis device 100 cleans blood by circulating the blood out of the body of a patient through: a blood circulating system including a dialyzer that cleans blood by allowing blood and dialysis fluid to come in to contact with each other through a semipermeable membrane, an arterial blood circuit that takes in blood from the patient and inputs the blood to the dialyzer, and a venous blood circuit that returns, to the patient, blood output from the dialyzer; a dialysis-fluid supplying and discharging system including lines that supply and discharge the dialysis fluid to and from the dialyzer; and a blood pump provided to the arterial blood circuit. The dialysis-fluid supply system 10 generates dialysis fluid by mixing a diluent and a plurality of drugs, and supplies the generated dialysis fluid to the dialysis device 100.

The dialysis-fluid supply system 10 has a relatively small-sized configuration suitable for medical treatment of a small number (for example, one) of patients, and is expected to be used in a relatively small-sized facility such as a hospital or a home, but not in a dialysis clinic. However, the capacities of tanks T1 and T2 to be described later can be changed to provide a configuration suitable for simultaneous medical treatment of a large number of patients and use at a large-sized facility. The dialysis-fluid supply system 10 and the dialysis device 100 are preferably integrated to achieve reduction in the size of the entire system, but may be separated.

The dialysis-fluid supply system 10 mainly includes the mixing tank T1 and the storage tank T2, a supply mechanism that supplies a drug and a diluent to the mixing tank T1, a transfer mechanism that transfers dialysis fluid generated by the mixing tank T1 to the storage tank T2, an output mechanism that outputs dialysis fluid stored in the storage tank T2 to the dialysis device 100, and a control unit 16 that controls driving of various mechanisms.

The mixing tank T1 is a container for generating dialysis fluid by mixing and diluting water and a drug. The mixing tank T1 includes a fluid amount sensor 18 that detects the amount of supplied fluid, such as a level switch that detects a fluid level, or a load sensor that detects the weight of supplied fluid. The mixing tank T1 desirably includes a concentration sensor (not illustrated) that detects the concentration of the dialysis fluid generated in the mixing tank T1.

The storage tank T2 is a container for storing the dialysis fluid generated by the mixing tank T1. The dialysis fluid generated by the mixing tank T1 is transferred to the storage tank T2 by a transfer mechanism to be described later. The dialysis fluid stored in the storage tank T2 is output to the dialysis device 100 at a constant speed by the output mechanism. The storage tank T2 desirably includes the fluid amount sensor 18 that detects the amount of stored fluid to detect overflow of dialysis fluid from the storage tank T2 and the amount of remaining dialysis fluid. However, the fluid amount sensor 18 may be omitted from the storage tank T2 when it is possible to accurately control timing of generation and use of dialysis fluid, as described later.

The capacities of the mixing tank T1 and the storage tank T2 are determined in accordance with a use limit time duration of dialysis fluid. In the present embodiment, the dialysis fluid is bicarbonate dialysis fluid. The bicarbonate dialysis fluid is, as is well known, obtained by mixing and diluting two kinds of drugs, which are a drug A and a drug B. The drug A includes an electrolyte component (for example, sodium chloride, potassium chloride, calcium chloride, magnesium chloride, or sodium acetate), a pH adjuster (for example, acetic acid), and sugar (for example, glucose). The drug B includes sodium bicarbonate.

The concentration of the bicarbonate dialysis fluid changes due to generation of a deposit when a certain time elapses after mixing of the drugs A and B, and thus a time duration in which the bicarbonate dialysis fluid is usable after its generation, in other words, the use limit time duration, is set. The use limit time duration differs slightly between kinds of product, but is typically two hours approximately. In the present embodiment, the mixing tank T1 has a capacity equal to or less than the amount of dialysis fluid generated and used in the use limit time duration. For example, when the use limit time duration is two hours and the flow rate of dialysis fluid used in dialysis treatment is 500 mL/min, the mixing tank T1 desirably has a capacity equal to or less than the amount of dialysis fluid used in the two-hour dialysis treatment, which is 500 mL×120 min=60 L. Since consideration needs to be given to such a problem that a time duration until a deposit is generated since the drugs A and B are mixed changes with solution temperature and the like, and to a time duration for which the dialysis fluid is stored after production, the use limit time duration is preferably set to be one hour to reliably prevent a deposit. In this case, when the flow rate of dialysis fluid is 500 mL/min, the mixing tank T1 desirably has a capacity equal to or less than 500 mL×60 min=30 L. Note that, of course, the capacity of the mixing tank T1 may be changed in accordance with the flow rate of dialysis fluid. Thus, for example, when the flow rate of dialysis fluid used in a dialysis treatment is 250 mL/min, the mixing tank T1 may have a maximum capacity equal to or less than 250 mL×60 min=15 L. Production of dialysis fluid needs to take at least 15 minutes to prevent insufficient dissolution of the drugs, and thus the mixing tank T1 desirably has a capacity equal to or more than the amount of dialysis fluid used in a dialysis treatment for 15 minutes. Specifically, the mixing tank T1 desirably has a capacity equal to or more than 7.5 L when the flow rate of dialysis fluid is 500 mL/min, or a capacity equal to or more than 3.75 L when the flow rate of dialysis fluid is the 250 mL/min.

The storage tank T2 desirably has a capacity larger than the capacity of the mixing tank T1 to prevent overflow of dialysis fluid from the storage tank T2. In order to prevent discontinuity of dialysis fluid output from the storage tank T2 in a dialysis treatment, the dialysis fluid needs to be transferred from the mixing tank T1 to the storage tank T2 before the storage tank T2 completely runs out of the dialysis fluid, in other words, while some dialysis fluid remains in the storage tank T2. Thus, to prevent the overflow of dialysis fluid from the storage tank T2, the storage tank T2 needs to have a capacity equal to or more than the sum of the capacity of the mixing tank T1 and the amount of remaining dialysis fluid.

The supply mechanism includes a water supply device 12 that supplies water as the diluent, a drug supply device 14 that supplies the drugs A and B, an input line Li connected with these devices, and a first input valve Vi1 provided in the input line Li. The water supply device 12 may have, but is not particularly limited to, any configuration capable of supplying highly pure water. Thus, the water supply device 12 may be, for example, an RO device that generates highly pure RO water by removing impurities from water through a reverse osmosis membrane (RO membrane), or a water treatment device that generates highly pure water through ion exchange resin and an ultrafiltration membrane (UF membrane). The water supply device 12 supplies water to the mixing tank T1 and the drug supply device 14 through the lines Lw and Li.

The drug supply device 14 supplies drugs for dialysis fluid to the mixing tank T1. In the present embodiment, since the dialysis fluid is bicarbonate dialysis fluid, the supplied drugs are the drugs A and B. The drugs A and B are set to the drug supply device 14 in advance, and supplied to the mixing tank T1 together with water. The drugs A and B set in the drug supply device 14 are desirably individually packaged in advance in an amount necessary for each generation of the dialysis fluid. When the dialysis-fluid supply system 10 includes a mechanism for measuring the drugs A and B, however, the drugs A and B do not need to be measured and packaged in advance.

The drugs A and B set in the drug supply device 14 may be provided in powder form or tablet form, or may be provided as concentrated solution dissolved with a small amount of water. Alternatively, one of the drugs A and B set in the drug supply device 14 may be in liquid form, and the other may be in solid form (powder form or tablet form). The drug supply device 14 may supply the drugs A and B set in powder form or tablet form directly to the mixing tank T1, or may supply the drugs A and B to the mixing tank T1 as a concentrated solution dissolved with a small amount of water. The water supply device 12 supplies water to the drug supply device 14 through the line Lw, and a concentrated solution obtained by dissolving the drugs A and B with water is supplied to the mixing tank T1. In any case, the drug supply device 14 only needs to supply a necessary amount of the drugs of the dialysis fluid to the mixing tank T1. In the present embodiment, the bicarbonate dialysis fluid is exemplarily described, but the dialysis fluid may be any other kind of dialysis fluid generated by mixing and diluting a plurality of drugs.

The water from the water supply device 12 and the drugs from the drug supply device 14 are output to the input line Li. The input line Li is a line that is connected with the mixing tank T1 and through which the drugs and the water are supplied to the mixing tank T1. The input line Li is provided with the first input valve Vi1, opening and closing of which is driven by the control unit 16.

The transfer mechanism transfers dialysis fluid generated by the mixing tank T1 to the storage tank T2, and includes a link line Lm, a second input valve Vi2, and a transfer pump 20. The link line Lm is a line that couples the mixing tank T1 and the storage tank T2 with each other and in which the transfer pump 20 and the second input valve Vi2 are provided. The control unit 16 drives the transfer pump 20 to transfer the dialysis fluid from the mixing tank T1 to the storage tank T2. During the transfer, the second input valve Vi2 is opened.

The link line Lm is connected with a circulation line Lr. The circulation line Lr is a line that couples the link line Lm and the input line Li with each other and in which a circulation valve Vr is provided. The fluid inside the mixing tank T1 is internally and externally circulated through the circulation line Lr to perform agitation of the drugs and the water. Specifically, the agitation of the drugs and the water is performed by driving the transfer pump 20 while the circulation valve Vr is opened and the first and second input valves Vi1 and Vi2 are closed. Accordingly, the fluid inside the mixing tank T1 returns to the mixing tank T1 through the link line Lm, the circulation line Lr, and the input line Li, and this circulation agitates the fluid inside the mixing tank T1. This can prevent insufficient dissolution and remaining dialysis drugs.

The output mechanism outputs dialysis fluid stored in the storage tank T2 to the dialysis device 100, and includes an output line Lo and an output valve Vo. The output line Lo is a line that couples the storage tank T2 and the dialysis device 100 with each other and in which the output valve Vo is provided. While the dialysis fluid is output, in other words, while a dialysis treatment is performed, the output valve Vo is kept opened. The output line Lo is connected with a discard line Ld. The discard line Ld is a line that couples the output line Lo and a drain with each other and in which a discard valve Vd is provided. Any dialysis fluid remaining after the dialysis treatment has ended and fluid (cleaning fluid and rinsing water) used to clean the tanks T1 and T2 are discarded through the discard line Ld.

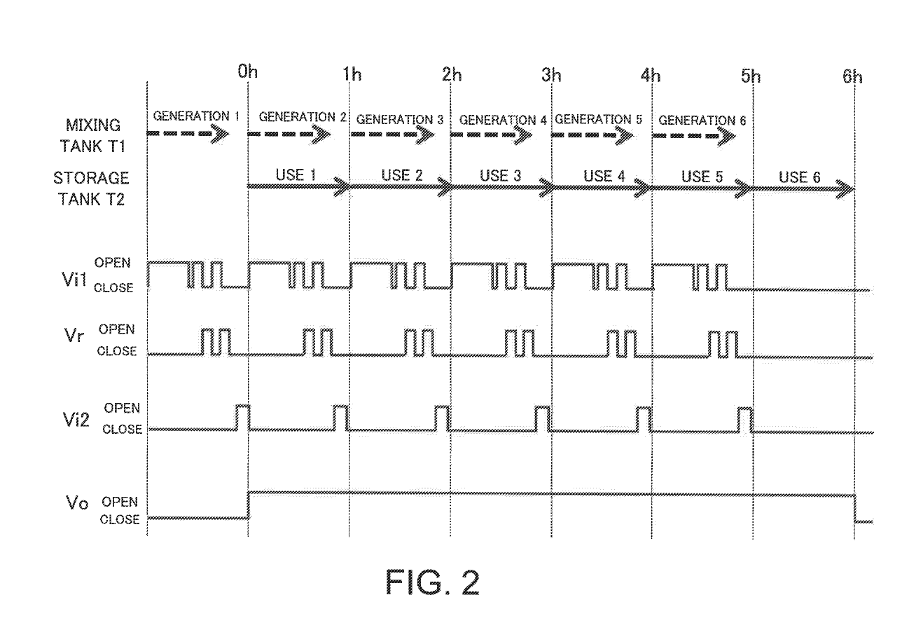

The control unit 16 controls driving of, for example, the water supply device 12, the drug supply device 14, and the valves Vi1, Vi2, Vo, and Vr described above. In the present embodiment, the control unit 16 switches opening and dosing of the valves to generate dialysis fluid in the mixing tank T1 while outputting dialysis fluid from the storage tank T2, and transfer the dialysis fluid in the mixing tank T1 to the storage tank T2 when the amount of dialysis fluid remaining in the storage tank T2 becomes low. FIG. 2 is a timing chart illustrating timing of the opening and closing of the valves Vi1, Vi2, Vo, and Vr when a dialysis treatment is continuously performed with the dialysis-fluid supply system 10 for six hours.

When a dialysis treatment is started, first, dialysis fluid is generated in the mixing tank T1. The generation of the dialysis fluid is achieved by sequentially performing supply of water to the mixing tank T1, supply of the drug B to the mixing tank T1, agitation, supply of the drug A to the mixing tank T1, and agitation. Specifically, first, the first input valve Vi1 is opened to supply a certain amount of water into the mixing tank T1. This supply amount of water is controlled based on a result of detection by the fluid amount sensor 18 provided to the mixing tank T1. Subsequently, while the first input valve Vi1 is opened, the drug B is supplied. Simultaneously, water is supplied together with the drug B, and this supply amount of water is controlled based on a result of detection by the fluid amount sensor 18. After the drug B is supplied, the transfer pump 20 is driven while the first input valve Vi1 is closed and the circulation valve Vr is opened, so as to circulate (agitate) the fluid inside the tank T1. Once the agitation is completed, the concentration of the fluid inside the mixing tank T1 is detected by the concentration sensor (not illustrated). If the detected concentration is within a predetermined reference range, the first input valve Vi1 is then opened and the circulation valve Vr is closed to supply the drug A to the mixing tank T1. The amount of water supplied together with the drug A is controlled based on a result of detection by the fluid amount sensor 18. After the drug A is supplied, the transfer pump 20 is driven again while the first input valve Vi1 is closed and the circulation valve Vr is opened, so as to circulate (agitate) the fluid inside the tank T1. Then, the concentration of the fluid inside the mixing tank T1 is detected by the concentration sensor again, and if the detected concentration has no problem, the generation of dialysis fluid is completed.

Once the dialysis fluid is generated in the mixing tank T1, the second input valve Vi2 is opened and the transfer pump 20 is driven to transfer the dialysis fluid from the mixing tank T1 to the storage tank T2. Once the dialysis fluid is transferred to the storage tank T2, a dialysis treatment is started. Specifically, the output valve Vo is opened to output the dialysis fluid stored in the storage tank T2 to the dialysis device 100. Until 6 h, at which the dialysis treatment is ended, the output valve Vo is kept opened to continuously transfer a certain amount of dialysis fluid to the dialysis device 100. As described above, the mixing tank T1 generates dialysis fluid in an amount used in a one-hour dialysis treatment. Thus, dialysis fluid (Use N) output between (N-1) h to Nh (N is an integer of one to six) in the storage tank T2 is dialysis fluid (Generation N) generated at the N-th time in the mixing tank T1.

After the generated dialysis fluid is transferred to the storage tank T2, the mixing tank T1 starts the second generation of dialysis fluid. Once the generation is completed, the generated dialysis fluid is transferred from the mixing tank T1 to the storage tank T2 if the remaining amount of the dialysis fluid inside the storage tank T2 becomes lower than a predetermined threshold. Then, the same processing is repeated to perform the generation and transfer of dialysis fluid a total of six times.

Before generation and outputting of dialysis fluid, the mixing tank T1 and the storage tank T2 are cleaned. At the time of cleaning, first, cleaning fluid is supplied and stored in the mixing tank T1 to clean the mixing tank T1. Subsequently, the cleaning fluid in the mixing tank T1 is transferred to the storage tank T2 and stored therein. Once the storage tank T2 is cleaned, the stored cleaning fluid is discharged to the drain. Subsequently, rinsing water is sequentially transferred to the mixing tank T1 and the storage tank T2 and then discharged to the drain.

As described above, in the present embodiment, the two tanks T1 and T2 are provided, the tank T1 generating dialysis fluid, and the tank T2 outputting the dialysis fluid. This configuration is employed for the following reasons.

In most conventional dialysis-fluid supply systems, dialysis fluid is continuously generated and continuously supplied. Specifically, in the conventional dialysis-fluid supply system, a concentrated solution of the drugs A and B, which is obtained in advance, and water, are accurately measured by a measurement pump before flowing into a line, and then mixed together in the line to generate dialysis fluid at a desired concentration. The measurement pump provided to this dialysis-fluid continuous supply system needs to be able to accurately measure the amount of fluid and transfer the fluid. However, typically, such a measurement pump is extremely expensive and needs frequent maintenance, and thus is difficult to use at a small-sized facility.

To solve this difficulty, a dialysis-fluid batch supply system has been proposed in which certain amounts of drugs and water are supplied to a tank to generate dialysis fluid. In such a dialysis-fluid batch supply system, when the tank includes a fluid amount sensor, no measurement pump is needed, thereby achieving reduction in the price of the entire system and also in maintenance work. However, only one tank that generates dialysis fluid is provided in the conventional dialysis-fluid batch supply system. Thus, the system could not generate a large amount of dialysis fluid.

As described above, the use limit time duration is set for the bicarbonate dialysis fluid. When the bicarbonate dialysis fluid is generated in one tank in excess of the use limit time duration, some of the dialysis fluid that has not been used in the use limit time duration is discarded. Thus, in a configuration in which dialysis fluid is generated in one tank only, part of the dialysis fluid in excess of an amount used in medical treatment in the use limit time duration (two hours approximately) cannot be output. Accordingly, when a dialysis-fluid supply system provided with one tank only is employed, a dialysis treatment can be continuously performed over a time duration less than the use limit time duration.

However, a dialysis treatment is continuously performed for a longer time duration depending on the condition and lifestyle of a patient. In particular, it has been desired to reduce the frequency of dialysis treatment by performing the dialysis treatment for a long time (for example, six hours) while the patient is sleeping. However, the conventional dialysis-fluid supply system provided with only one tank could not sufficiently meet such a demand by patients.

To solve this problem, in the present embodiment, as described above, the two tanks T1 and T2 are provided, the mixing tank T1 generating dialysis fluid, and the storage tank T2 storing the generated dialysis fluid. This configuration allows continuous outputting of the dialysis fluid generated within the use limit time, without discontinuity. Accordingly, the freedom of medical treatment time can be increased in accordance with the condition and lifestyle of a patient. In the present embodiment, since the mixing tank T1 includes the fluid amount sensor 18, a pump that transfers, for example, water does not need to have a measurement function, which leads to reduction in the price of the entire system and also in the amount of maintenance work.

Patent Literature 3 discloses a technology in which two tanks are provided such that a tank that generates dialysis fluid and a tank that outputs the dialysis fluid are sequentially switched. In this technology too, dialysis fluid generated within a use limit time can be continuously output without discontinuity. In the technology of Patent Literature 3, however, each tank functions as the dialysis-fluid generation tank and the dialysis-fluid outputting tank. Accordingly, the technology of Patent Literature 3 requires, for the two tanks, two sets of various valves and sensors necessary for functioning as the dialysis-fluid generation tank and various valves and sensors necessary for functioning as the dialysis-fluid storage tank. In the present embodiment, the tank T1, as one of the tanks, has a dialysis-fluid generation function only, and the other tank T2 has a dialysis-fluid outputs function only. This configuration only requires, for the one tank, one set of various valves and sensors necessary for functioning as the dialysis-fluid generation tank, and only requires, for the other tank, one set of various valves and sensors necessary for functioning as the dialysis-fluid storage tank. Accordingly, the present embodiment can achieve further reduction in cost of the entire system with a simpler configuration.

The above-described configuration is merely exemplary. Modifications of the configuration are possible as appropriate to achieve a configuration including the mixing tank T1, which generates dialysis fluid, and the storage tank T2, which stores therein and outputs the dialysis fluid generated by the mixing tank. For example, in the present embodiment, the drugs (drugs A and B) are automatically supplied by the drug supply device 14, but may be manually supplied. In the present embodiment, the transfer pump 20 is provided to transfer dialysis fluid from the mixing tank T1 to the storage tank T2, but a height difference between the tanks may be exploited to transfer the dialysis fluid without the pump. In the above-described embodiment, fluid inside the mixing tank T1 is agitated by being internally and externally circulated. However, the agitation is not limited to this method but may be performed by another method. For example, a bladed wheel for agitation may be provided in each tank to perform the agitation.

### REFERENCE SIGNS LIST

1 Dialysis system

10 Dialysis-fluid supply system

12 Water supply device

14 Drug supply device

16 Control unit

18 Fluid amount sensor

20 Transfer pump

100 Dialysis device

Ld Discard line

Li Input line

Lm Link line

Lo Output line

Lr Circulation line

T1 Mixing tank

T2 Storage tank

Vd Discard valve

Vi1 First input valve

Vi2 Second input valve

Vo Output valve

Vr Circulation valve

1. A dialysis-fluid supply system that generates dialysis fluid by mixing a diluent and at least two drugs and outputs the dialysis fluid, the system comprising:
a mixing tank that generates dialysis fluid by mixing the drugs and the diluent;
a storage tank that stores therein the dialysis fluid generated by the mixing tank and outputs the dialysis fluid;
a transfer mechanism that transfers the dialysis fluid generated by the mixing tank to the storage tank;
an output mechanism that outputs the dialysis fluid stored in the storage tank to a dialysis device; and
a control unit that controls driving of the transfer mechanism and the output mechanism.

2. The dialysis-fluid supply system according to claim 1, wherein the control unit causes the mixing tank to generate dialysis fluid while causing the storage tank to output dialysis fluid to a dialysis device.

3. The dialysis-fluid supply system according to claim 1, wherein
the mixing tank includes a weight sensor that measures the weight of supplied fluid or a level sensor that measures the level of supplied fluid, and
the control unit monitors the supply amount of each drug or the diluent to the mixing tank based on a result of detection by the weight sensor or the level sensor.

4. The dialysis-fluid supply system according to claim 1, further comprising a circulation mechanism that internally and externally circulates fluid inside the mixing tank to perform agitation.

5. The dialysis-fluid supply system according to claim 1, wherein the capacity of the storage tank is larger than the capacity of the mixing tank.

6. The dialysis-fluid supply system according to claim 1, wherein the control unit executes a cleaning process of cleaning the mixing tank and the storage tank by causing cleaning fluid to flow through the mixing tank and the storage tank in this order.

7. The dialysis-fluid supply system according to claim 1, wherein
the dialysis-fluid supply system is a personal dialysis-fluid supply system for use in a medical treatment of one patient, and
each of the at least two drugs is in powder form and is individually packaged in an amount necessary for a single generation of the dialysis fluid, prior to use,
the dialysis-fluid supply system further comprising:
a drug supply device that supplies all of the at least two drugs individually packaged and set to the mixing tank without measuring the at least two drugs.

8. The dialysis-fluid supply system according to claim 1, wherein
the dialysis-fluid supply system is a personal dialysis-fluid supply system for use in a medical treatment of one patient, and
an amount of dialysis fluid generated in a single generation process in each tank is, assuming that a flow rate of dialysis fluid used is A L/min, a time duration necessary for the generation process is B min, and a use limit time duration of the dialysis fluid is C min, is equal to or greater than B×A and is equal to or smaller than C×A.

9. The dialysis-fluid supply system according to claim 8, wherein
the mixing tank has a capacity of 3.75 L or greater and 30 L or smaller.

10. The dialysis-fluid supply system according to claim 1, further comprising:
a discard line through which dialysis fluid remaining in the storage tank after completion of a dialysis treatment and fluid used to clean the storage tank is discarded from the storage tank.

11. The dialysis-fluid supply system according to claim 1, wherein
the at least two drugs comprise a drug A including an electrolyte component and a pH adjuster, and a drug B including sodium bicarbonate, and
after the drug B is supplies to the mixing tank and agitated, the drug A is further supplied to the mixing tank and agitated.
